(19) United States
(12) Patent Application Publication
(10) Pub. No.: US 2025/0308658 A1
(43) Pub. Date: Oct. 2, 2025

(54) MEDICAL RECORD SUPPORT APPARATUS, MEDICAL RECORD SUPPORT SYSTEM, AND MEDICAL RECORD SUPPORT METHOD

(71) Applicants: NEC Corporation, Tokyo (JP); NATIONAL CENTER FOR GLOBAL HEALTH AND MEDICINE, Tokyo (JP)

(72) Inventors: Takafumi YOSHINAGA, Tokyo (JP); Yasushi OKADA, Tokyo (JP); Takumi TANAKA, Tokyo (JP); Shigeki KANO, Tokyo (JP); Shohei YAMAGUCHI, Tokyo (JP); Kengo MIYO, Tokyo (JP); Ryota NISHI, Tokyo (JP)

(73) Assignees: NEC Corporation, Tokyo (JP); NATIONAL CENTER FOR GLOBAL HEALTH AND MEDICINE, Tokyo (JP)

(21) Appl. No.: 19/092,478

(22) Filed: Mar. 27, 2025

(30) Foreign Application Priority Data
Mar. 28, 2024 (JP) 2024-052727

(51) Int. Cl. G16H 10/65; G16H 40/67

(52) U.S. Cl. CPC G16H 10/65; G16H 40/67

(57) ABSTRACT

An example object of the present disclosure is to improve efficiency in creating a medical record based on a medical interview. A medical record support apparatus according to the present disclosure includes: a first acquisition unit configured to acquire text information of an utterance content converted by speech recognition in which speakers are distinguished for conversational speech data between a medical worker and a patient during a medical interview; a generation unit configured to generate an instruction sentence for a language model to be output in a medical record format from the text information; and a second acquisition unit configured to acquire medical record information of the patient by inputting the instruction sentence to the language model.

### INCORPORATION BY REFERENCE

This application is based upon and claims the benefit of priority from Japanese patent application No.2024-052727, filed on Mar. 28, 2024, the disclosure of which is incorporated herein in its entirety by reference.

### TECHNICAL FIELD

The present disclosure relates to a medical record support apparatus, a medical record support system, a medical record support method, and a medical record support program.

### BACKGROUND ART

At a medical site, it is necessary for a medical worker such as a doctor or a nurse to perform a medical interview with a patient and create a medical record. Further, for example, it is common that a nurse serves a large number of common patients together with other nurses. Therefore, a nursing record that is an example of the medical record related to a specific patient is shared with other nurses and medical workers.

Japanese Unexamined Patent Application Publication No. 2023-026640 discloses a technology related to processing of creating an electronic medical record from speech data of a conversation between a doctor and a patient.

### SUMMARY

Here, there is a problem that a burden of creating a medical record is large for a medical worker. The reason for the burden is that a medical worker is busy in serving a large number of patients, and a lot of time is spent on work of creating the medical record. Therefore, it is difficult for the medical worker to spend time on original work (examination, nursing, treatment, and the like).

An example object of the present disclosure is to provide a medical record support apparatus, a medical record support system, a medical record support method, and a medical record support program for improving efficiency in creating a medical record based on a medical interview.

In a first example aspect, a medical record support apparatus includes:
a first acquisition unit configured to acquire text information of an utterance content converted by speech recognition in which speakers are distinguished for conversational speech data between a medical worker and a patient during a medical interview;
a generation unit configured to generate an instruction sentence for a language model to be output in a medical record format from the text information; and
a second acquisition unit configured to acquire medical record information of the patient by inputting the instruction sentence to the language model.

In a second example aspect, a medical record support system includes:
an eyewear-type wearable terminal worn by a medical worker during a medical interview; and
an information processing apparatus communicably connected to the eyewear-type wearable terminal, in which
the information processing apparatus includes:
a first acquisition unit configured to acquire text information of an utterance content converted by speech recognition in which speakers are distinguished for conversational speech data between the medical worker and a patient during the medical interview, the conversational speech data being collected by the eyewear-type wearable terminal;
a generation unit configured to generate an instruction sentence for a language model to be output in a medical record format from the text information; and
a second acquisition unit configured to acquire medical record information of the patient by inputting the instruction sentence to the language model, and
the eyewear-type wearable terminal is configured to display the medical record information acquired by the second acquisition unit on a wearer screen.

In a third example aspect, a medical record support method executed by a computer includes:
acquiring text information of an utterance content converted by speech recognition in which speakers are distinguished for conversational speech data between a medical worker and a patient during a medical interview;
generating an instruction sentence for a language model to be output in a medical record format from the text information; and
acquiring medical record information of the patient by inputting the instruction sentence to the language model.

In a fourth example aspect, a medical record support program causes a computer to perform:
first acquisition processing of acquiring text information of an utterance content converted by speech recognition in which speakers are distinguished for conversational speech data between a medical worker and a patient during a medical interview;
generation processing of generating an instruction sentence for a language model to be output in a medical record format from the text information; and
second acquisition processing of acquiring medical record information of the patient by inputting the instruction sentence to the language model.

An example advantage according to the above-described embodiments is efficient creation of a medical record based on a medical interview.

### BRIEF DESCRIPTION OF DRAWINGS

The above and other aspects, features and advantages of the present disclosure will become more apparent from the following description of certain exemplary embodiments when taken in conjunction with the accompanying drawings, in which:

FIG. 1 is a block diagram illustrating a configuration of a medical record support apparatus according to the present disclosure;

FIG. 2 is a flowchart illustrating a flow of a medical record support method according to the present disclosure;

FIG. 3 is a block diagram illustrating an overall configuration of a medical record support system according to the present disclosure;

FIG. 4 is a block diagram illustrating a configuration of augmented reality (AR) glasses according to the present disclosure;

FIG. 5 is a block diagram illustrating a hardware configuration of the AR glasses according to the present disclosure;

FIG. 6 is a block diagram illustrating a configuration of a medical record support server according to the present disclosure;

FIG. 7 is a block diagram illustrating a hardware configuration of the medical record support server according to the present disclosure;

FIG. 8 is a sequence chart illustrating a flow of medical record support processing according to the present disclosure;

FIG. 9 is a sequence chart illustrating a flow of the medical record support processing according to the present disclosure;

FIG. 10 is a sequence chart illustrating a flow of the medical record support processing according to the present disclosure;

FIG. 11 is a diagram for describing a display example of a wearer screen of the AR glasses at the start of the medical record support processing according to the present disclosure and a concept of a start operation;

FIG. 12 is a diagram illustrating a display example of the wearer screen of the AR glasses immediately after the start of the medical record support processing (update mode) according to the present disclosure;

FIG. 13 is a diagram for describing a medical interview with a patient by a medical worker and a concept of an operation of ending the medical record support processing according to the present disclosure;

FIG. 14 is a diagram illustrating a display example of the wearer screen of the AR glasses during speech recognition of the medical record support processing (update mode) according to the present disclosure;

FIG. 15 is a diagram illustrating an example of text conversion and a generated prompt based on a speech recognition result according to the present disclosure;

FIG. 16 is a diagram illustrating a display example of the latest medical record output by a large language model (LLM) from the generated prompt according to the present disclosure; and

FIG. 17 is a block diagram illustrating a configuration of the AR glasses according to the present disclosure.

### EXAMPLE EMBODIMENTS

Hereinafter, embodiments of the present disclosure will be described in detail with reference to the drawings. In the drawings, the same or corresponding elements are denoted by the same reference numerals, and repeated description is omitted as necessary for clarity of description.

### First Embodiment

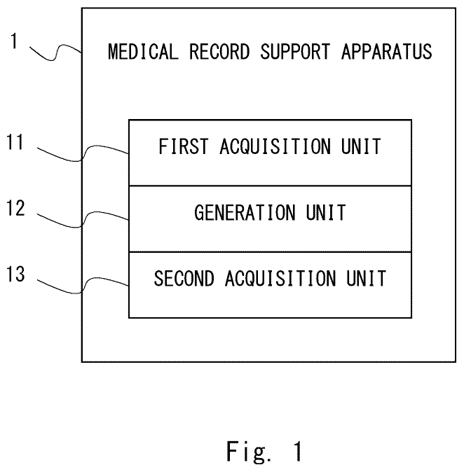

FIG. 1 is a block diagram illustrating a configuration of a medical record support apparatus 1. The medical record support apparatus 1 is an information processing apparatus for supporting work of creating a medical record by a medical worker using conversational speech data between the medical worker and a patient during a medical interview. Here, the “medical worker” is, for example, a doctor, a nurse, a physical therapist, or any other person qualified to engage in medical care. In addition, the “medical record” is a medical record created by a doctor, a nursing record created by a nurse, and other records related to medical treatment of a patient by a medical worker.

The medical record support apparatus 1 includes a first acquisition unit 11, a generation unit 12, and a second acquisition unit 13. The first acquisition unit 11 acquires text information of an utterance content converted by speech recognition in which speakers are distinguished for the conversational speech data between the medical worker and the patient during the medical interview.

The generation unit 12 generates an instruction sentence for a language model to be output in a medical record format from the text information acquired by the first acquisition unit 11. Here, the “language model” is a computer program or an information system that receives text data in which a question or an instruction is expressed in a natural language, and outputs text data generated, converted, processed, summarized, or the like by predetermined computation for the input text data. The language model corresponds to a natural language model in an artificial intelligence (AI) model. The “instruction sentence” is text data including one or more sentences. The “instruction sentence” is input data for the language model, and includes at least a part of the text information acquired by the first acquisition unit 11. Furthermore, the “instruction sentence” is a sentence in a format for causing the language model to output the text information in the medical record format.

The second acquisition unit 13 acquires medical record information of the patient by inputting the instruction sentence generated by the generation unit 12 to the language model. That is, the language model generates and outputs the text information of the medical record information by converting, processing, summarizing, and the like the text information included in the instruction sentence according to the input instruction sentence in the medical record format designated by the instruction sentence. Therefore, the second acquisition unit 13 acquires the text data output from the language model to which the instruction sentence has been input, as the medical record information of the patient who has undergone the medical interview.

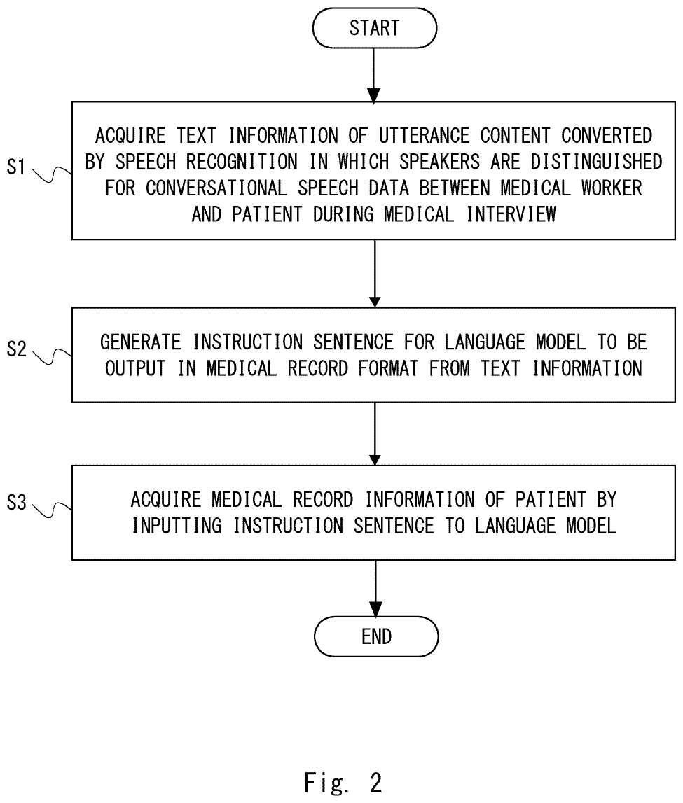

FIG. 2 is a flowchart illustrating a flow of a medical record support method. First, the first acquisition unit 11 acquires the text information of the utterance content converted by the speech recognition in which the speakers are distinguished for the conversational speech data between the medical worker and the patient during the medical interview (S1). Next, the generation unit 12 generates the instruction sentence for the language model to be output in a medical record format from the text information (S2). Then, the second acquisition unit 13 acquires the medical record information of the patient by inputting the instruction sentence to the language model (S3).

As described above, the medical record support apparatus 1 acquires the medical record information in the medical record format by using each utterance content of the medical worker and the patient during the medical interview with the patient by the medical worker. Therefore, the medical worker can obtain the medical record information based on the medical interview by using the medical record support apparatus 1 when verbally performing the medical interview with the patient. Therefore, it is possible to efficiently create the medical record based on the medical interview.

The medical record support apparatus 1 includes a processor, a memory, and a storage device as components (not illustrated). Furthermore, the storage device stores, for example, a computer program in which processing of the medical record support method of FIG. 2 is implemented. Then, the processor reads the computer program or the like from the storage device into the memory and executes the computer program. As a result, the processor implements the functions of the first acquisition unit 11, the generation unit 12, and the second acquisition unit 13.

Alternatively, each component of the medical record support apparatus 1 may be implemented by dedicated hardware. In addition, some or all of the components of each apparatus may be implemented by general-purpose or dedicated circuitry, a processor, or a combination thereof. These components may be implemented by a single chip or may be implemented by a plurality of chips connected via a bus. Some or all of the components of each apparatus may be implemented by a combination of the above-described circuitry or the like and a program. Furthermore, as the processor, a central processing unit (CPU), a graphics processing unit (GPU), a field-programmable gate array (FPGA), a quantum processor (quantum computer control chip), or the like can be used.

Furthermore, in a case where some or all of the components of the medical record support apparatus 1 are implemented by a plurality of information processing apparatuses, circuitries, and the like, the plurality of information processing apparatuses, circuitries, and the like may be arranged in a centralized manner or in a distributed manner. For example, the information processing apparatuses, the circuitries, or the like may be implemented in the form of a client server system, a cloud computing system, or the like in which the information processing apparatuses, the circuitries, or the like are connected to each other via a communication network. Furthermore, the function of the medical record support apparatus 1 may be provided in a software as a service (SaaS) format.

### Second Embodiment

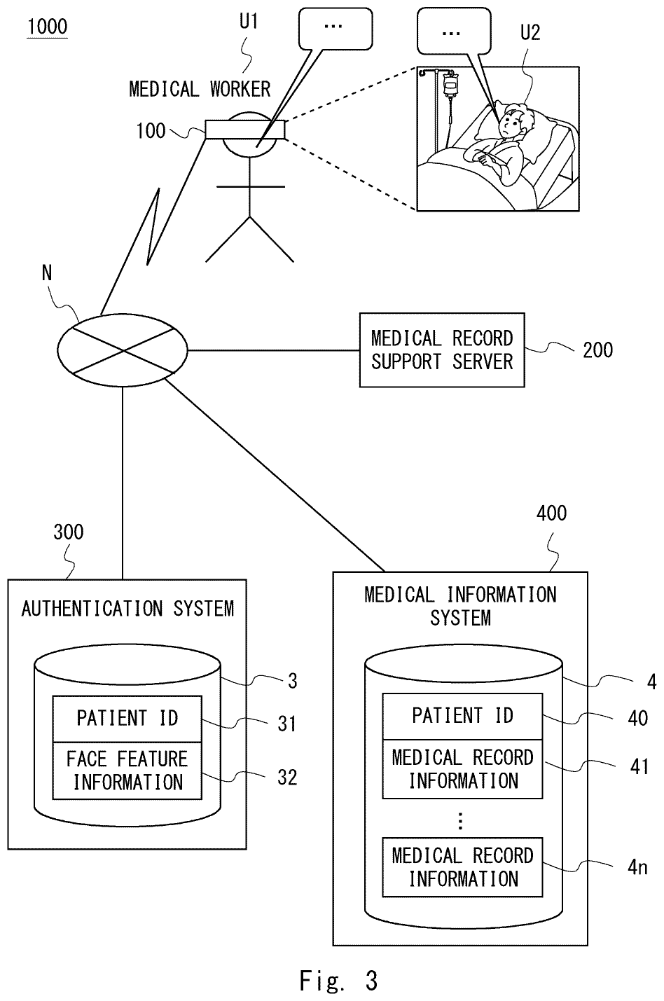

FIG. 3 is a block diagram illustrating an overall configuration of a medical record support system 1000. The medical record support system 1000 is an information system for supporting a medical worker U1 to create a medical record of a patient U2 through a medical interview with the patient U2 by the medical worker U1 wearing augmented reality (AR) glasses 100. The medical record support system 1000 includes the AR glasses 100, a medical record support server 200, an authentication system 300, and a medical information system 400. However, the medical record support system 1000 may include at least the AR glasses 100 and the medical record support server 200. The AR glasses 100, the medical record support server 200, the authentication system 300, and the medical information system 400 are communicably connected via a network N. Here, the network N is a wired or wireless communication line.

As described above, the medical worker U1 is a doctor, a nurse, or the like. The medical worker U1 performs the medical interview with the patient U2 in a state of wearing the AR glasses 100 according to the present disclosure. The patient U2 is an inpatient on a hospital bed or a patient who visits a hospital and receives the medical interview from the medical worker U1. In a case where the medical worker U1 is a nurse, medical record information is electronic data of a nursing record. In a case where the medical worker U1 is a doctor, the medical record information is electronic data of the medical record. Then, the medical record information includes the medical record and the nursing record, and may be a so-called “electronic medical record”

The authentication system 300 is an information system that performs authentication processing for identity confirmation of the patient. The authentication system 300 performs face authentication in biometric authentication as an example of the identity confirmation. The authentication system 300 may perform biometric authentication other than the face authentication or identity confirmation processing other than the biometric authentication. The authentication system 300 includes at least an authentication database (DB) 3. The authentication DB 3 is a database that manages a patient ID 31 and face feature information 32 in association with each other. The authentication DB 3 may manage attribute information (a name or the like) of the patient other than the face feature information 32 in association with the patient ID 31. The patient ID 31 is identification information of the patient U2. The face feature information 32 is a feature vector including a set of feature points of the face of the patient corresponding to the patient ID 31. For example, the face feature information 32 is a set of pieces of information calculated using, as a feature amount, a distance or the like between a plurality of feature points indicating face features (the eyes, the nose, the mouth, and the like) in a face region detected from a face image of the corresponding patient.

The authentication system 300 determines whether the face authentication has succeeded or failed based on a matching degree between face feature information (query) based on a face image included in a face authentication request received via the network N and a plurality of pieces of (master) face feature information 32 in the authentication DB 3. In a case where the face authentication has succeeded, the authentication system 300 specifies the patient ID 31 (and the attribute information) associated with the face feature information 32 for which the face authentication has succeeded, includes the specified patient ID 31 (and the specified attribute information) in a face authentication result, and transmits the face authentication result as a response to a request source.

The authentication system 300 may further include an iris authentication DB for iris authentication of the medical worker. In this case, the iris authentication DB manages a medical worker ID and iris feature information in association with each other. The iris authentication DB may manage attribute information (for example, qualification information, names, and the like of a doctor, a nurse, and the like) of the medical worker other than the iris feature information in association with the medical worker ID. Then, the authentication system 300 determines whether the iris authentication has succeeded or failed according to a matching degree between iris feature information (query) based on an iris image included in an iris authentication request received via the network N and a plurality of pieces of (master) iris feature information in the iris authentication DB. In a case where the iris authentication has succeeded, the authentication system 300 specifies the medical worker ID (and the attribute information) associated with the iris feature information for which the iris authentication has succeeded, includes the specified medical worker ID (and the specified attribute information) in an iris authentication result, and transmits the iris authentication result as a response to a request source.

The medical information system 400 is an information system for managing medical information such as the medical record. The medical information system 400 includes at least a medical record DB 4. The medical record DB 4 is a database that manages the medical record information of a patient. The medical record DB 4 manages a patient ID 40 and pieces of medical record information 41 to 4n (n is a natural number of 1 or more) in association with each other. The patient ID 40 is information equivalent to the patient ID 31. Therefore, the patient ID 31 of the specific patient U2 is the same as the patient ID 40 or information that can uniquely specify the patient ID 40.

The medical information system 400 searches the medical record DB 4 by using a patient ID included in a medical record information acquisition request received via the network N as a search key, and transmits the searched medical record information 41 or the like as a response to a request source.

The AR glasses 100 are an eyewear-type wearable terminal including a screen which also serves as a lens and through which light from an external peripheral space is transmitted. In addition, the AR glasses 100 display various types of display information described below on a field-of-view region of a real space visually recognized by the wearer through light transmitted from the outside, in a superimposed manner. The AR glasses 100 may be, for example, a head mounted display (HMD). The AR glasses 100 are an example of an eyewear-type wearable terminal worn by the medical worker U1 during the medical interview.

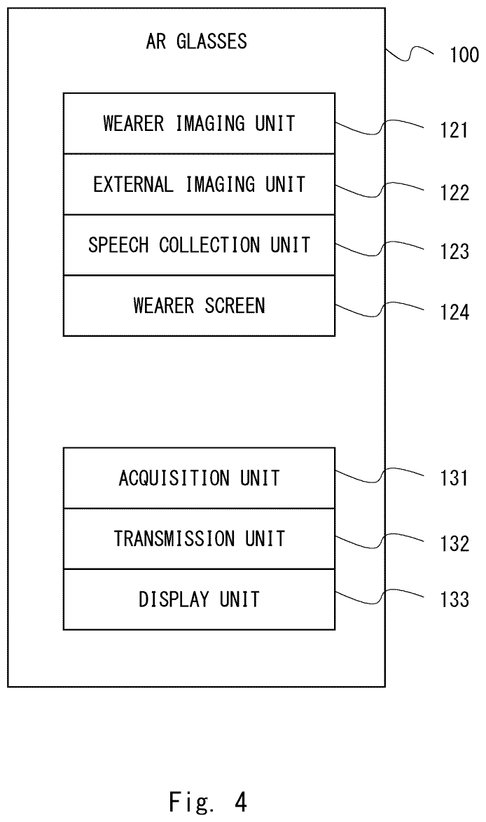

FIG. 4 is a block diagram illustrating a configuration of the AR glasses 100. The AR glasses 100 include a wearer imaging unit 121, an external imaging unit 122, a speech collection unit 123, a wearer screen 124, an acquisition unit 131, a transmission unit 132, and a display unit 133.

The wearer imaging unit 121 is a camera that images a region including the iris of the eye of the medical worker U1 who is a wearer, a circuitry or software that controls the camera, or the like. The wearer imaging unit 121 outputs the iris image obtained by imaging the iris of the medical worker U1 to the acquisition unit 131.

The external imaging unit 122 is a camera that images an imaging region including a display region (field-of-view region) of the wearer screen 124, a circuitry or software that controls the camera, or the like. The external imaging unit 122 images at least an area in front of the medical worker U1 who is the wearer, and outputs the captured image to the acquisition unit 131. The external imaging unit 122 may record the captured image in an internal storage unit (not illustrated).

The speech collection unit 123 is a microphone that collects a speech around the AR glasses 100, a circuitry or software that controls the microphone, or the like. The speech collection unit 123 collects a speech uttered by the medical worker U1 who is the wearer and a speech uttered by the patient U2. Therefore, the speech collection unit 123 collects a speech of a conversation between the medical worker U1 and the patient U2 present in the vicinity of the AR glasses 100 during the medical interview. The speech collection unit 123 may record collected speech information, at least speech information, in the storage unit.

The wearer screen 124 is a display that covers at least a part of the field-of-view region of the medical worker U1 who is the wearer of the AR glasses 100. In addition, the wearer screen 124 displays a display image (video) output by the display unit 133 in a superimposed manner in the field-of-view region through which light from an external peripheral space is transmitted.

The acquisition unit 131 acquires the iris image of the medical worker U1 from the wearer imaging unit 121, the face image of the patient U2 from the external imaging unit 122, and conversational speech data between the medical worker U1 and the patient U2 from the speech collection unit 123. The acquisition unit 131 is not limited to acquiring the conversational speech data, and may acquire speech data uttered by the medical worker U1 alone from the speech collection unit 123.

The transmission unit 132 may transmit the iris authentication request including the iris image of the medical worker U1 acquired by the acquisition unit 131 to the authentication system 300 via the network N. Then, the acquisition unit 131 acquires the iris authentication result from the authentication system 300 via the network N. In a case where the iris authentication result indicates success, the acquisition unit 131 acquires the medical worker ID (and the attribute information) included in the iris authentication result.

In addition, the transmission unit 132 may transmit the face authentication request including the face image of the patient U2 acquired by the acquisition unit 131 to the medical record support server 200 via the network N. Then, the acquisition unit 131 acquires the past medical record information of the patient U2 acquired from the medical information system 400 based on the face authentication result of the authentication system 300 in the medical record support server 200.

Furthermore, the transmission unit 132 transmits the conversational speech data acquired by the acquisition unit 131 to the medical record support server 200 via the network N. Then, the acquisition unit 131 acquires text information of an utterance content converted by speech recognition in which speakers are distinguished for the conversational speech data between the medical worker U1 and the patient U2 during the medical interview. Therefore, the acquisition unit 131 can also be said to be an example of the first acquisition unit 11 described above. In particular, the acquisition unit 131 may acquire text information converted in the medical record support server 200 based on the conversational speech data. Furthermore, the acquisition unit 131 may acquire text information converted in the medical record support server 200 based on uttered speech data of the medical worker U1 before and after the medical interview.

Furthermore, the acquisition unit 131 acquires, from the medical record support server 200, the medical record information output by inputting an instruction sentence to be output in the medical record format from the text information of the utterance content to a large language model described below, in the medical record support server 200. Therefore, the acquisition unit 131 can also be said to be an example of the second acquisition unit 13 described above. In this regard, in a case where the AR glasses have a function of a generation unit 224 in the medical record support server 200 described below, the AR glasses 100 can be an example of the medical record support apparatus 1 described above.

The display unit 133 may display the past medical record information of the patient U2 acquired by the acquisition unit 131 on the wearer screen 124. Furthermore, the display unit 133 may display the text information of the utterance content acquired by the acquisition unit 131 on the wearer screen 124. In addition, the display unit 133 displays the medical record information of the patient U2 acquired by the acquisition unit 131 on the wearer screen 124. In addition, the display unit 133 displays, on the wearer screen 124, the medical record information of the patient U2 acquired by the acquisition unit 131 and generated based on the uttered speech data of the medical interview.

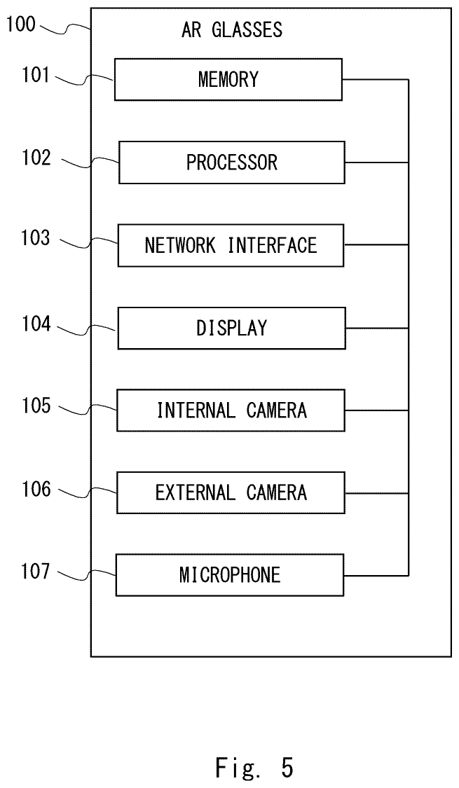

FIG. 5 is a block diagram illustrating a hardware configuration of the AR glasses 100. The AR glasses 100 include a memory 101, a processor 102, a network interface 103, a display 104, an internal camera 105, an external camera 106, and a microphone 107.

The memory 101 is implemented by a combination of a volatile memory and a nonvolatile memory. The volatile memory is, for example, a volatile storage device such as a random access memory (RAM), and is a storage region for temporarily holding information during operation of the processor 102. The nonvolatile memory is, for example, a nonvolatile storage device such as a flash memory. The memory 101 stores at least a computer program in which a part of processing of a medical record support method of the AR glasses 100 according to the present disclosure is implemented.

The processor 102 is a control apparatus that controls each component of the AR glasses 100. The processor 102 reads and executes software (computer program) from the memory 101. Therefore, the processor 102 implements the functions of the acquisition unit 131, the transmission unit 132, and the display unit 133. That is, the processor 102 performs a part of the processing of the medical record support method according to the present disclosure. The processor 102 may be, for example, a microprocessor, a multi processing unit (MPU), or a CPU. Further, the processor 102 may include a plurality of processors.

The network interface 103 may be used to communicate with network nodes. The network interface 103 may include, for example, a network interface card (NIC) conforming to IEEE 802.3 series. IEEE represents Institute of Electrical and Electronics Engineers. Furthermore, the network interface 103 may include a wireless local area network (LAN), a wired LAN, Wi-Fi (registered trademark), Bluetooth (registered trademark), and the like.

The display 104 is a display apparatus that displays information instructed from the processor 102. The display 104 is, for example, a screen such as a liquid crystal display or an organic electro-luminescence (EL) display. In particular, the display 104 transmits light from the outside and corresponds to the above-described wearer screen 124.

In response to an instruction from the processor 102, the internal camera 105 images a region including the iris of the eye of the wearer of the AR glasses 100, and outputs the captured iris image to the processor 102. The internal camera 105 corresponds to the above-described wearer imaging unit 121.

In response to an instruction from the processor 102, the external camera 106 images an external peripheral space including an area in front of the AR glasses 100, and outputs the captured image to the processor 102. The external camera 106 is, for example, a charge coupled device (CCD) image sensor, a complementary metal oxide semiconductor (CMOS) sensor, or the like. The external camera 106 includes one or more imaging devices. The external camera 106 corresponds to the above-described external imaging unit 122.

The microphone 107 is a sound collector that collects sound around the AR glasses 100.

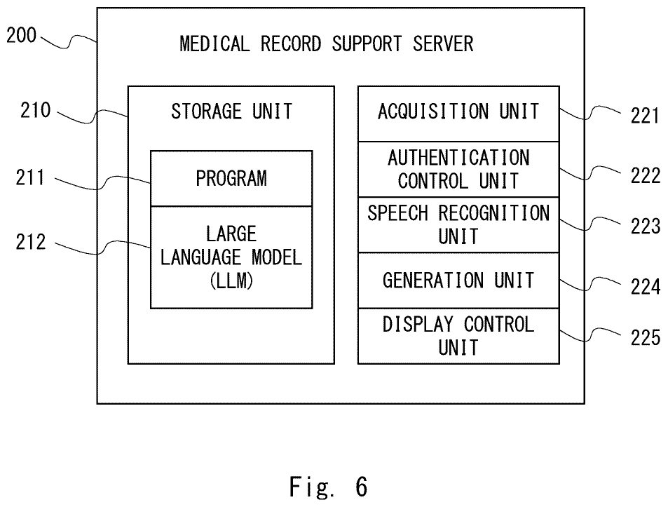

FIG. 6 is a block diagram illustrating a configuration of the medical record support server 200. The medical record support server 200 is an information processing apparatus for supporting creation of the medical record information of the patient U2 who has undergone the medical interview by the medical worker U1 by using the authentication system 300, the medical information system 400, and the like according to information received from the AR glasses 100. The medical record support server 200 is an example of the medical record support apparatus 1 described above. The medical record support server 200 includes a storage unit 210, an acquisition unit 221, an authentication control unit 222, a speech recognition unit 223, a generation unit 224, and a display control unit 225.

The storage unit 210 includes, for example, a nonvolatile storage device such as a hard disk or a flash memory, and a memory such as a RAM, that is, a volatile storage device. The storage unit 210 stores a program 211 and a large language model (LLM) 212. The program 211 is a computer program in which at least a part of medical record support processing according to the present disclosure is implemented. The LLM 212 is an example of the language model described above. The LLM 212 is a trained model that is trained by repeating deep learning using a large data set on a natural language model. The LLM 212 is a natural language model in which the number of times the deep learning is performed, the number of data sets used for learning, and the number of parameters to be learned are larger than those at the beginning when an AI model is started to spread. The LLM 212 uses the instruction sentence described in a specific format and including the text information to be processed as an input, performs computation on the text information included in the instruction sentence according to the instruction sentence, and outputs text data that is a computation result. Specifically, the LLM 212 converts, processes, summarizes, and the like the text information included in the instruction sentence in the medical record format designated in the instruction sentence, thereby generating and outputting the text information of the medical record information.

The acquisition unit 221 is an example of the first acquisition unit 11 and the second acquisition unit 13 described above. The acquisition unit 221 acquires the text information of the utterance content converted by the speech recognition in which the speakers are distinguished for the conversational speech data between the medical worker U1 and the patient U2 during the medical interview collected by the AR glasses 100. Here, the AR glasses 100 are an eyewear-type wearable terminal worn by the medical worker U1 during the medical interview. In addition, the acquisition unit 221 acquires, for the conversational speech data, the text information converted from a recognition result of the speech recognition in which the speakers are distinguished by the speech recognition unit 223 described below. Here, the text information may include, in addition to the speech recognition result from the conversational speech data, the text information converted as the utterance content of the medical worker U1 by speech recognition for the uttered speech data of the medical worker U1 before and after the medical interview.

In addition, the acquisition unit 221 acquires the medical record information of the patient U2 by inputting the instruction sentence generated by the generation unit 224 described below to the LLM 212. Furthermore, in a case where the face authentication result of the patient U2 received by the authentication control unit 222 described below indicates success, the acquisition unit 221 transmits the medical record information acquisition request to the medical information system 400, the medical record information acquisition request including the patient ID included in the face authentication result. Then, the acquisition unit 221 acquires the medical record information of the patient ID by receiving the medical record information corresponding to the patient ID included in the acquisition request from the medical information system 400. Then, the acquisition unit 221 transmits the acquired medical record information to the AR glasses 100.

The authentication control unit 222 controls the iris authentication of the medical worker U1 and the face authentication of the patient U2 by using the authentication system 300. Specifically, the authentication control unit 222 transmits the iris authentication request received from the AR glasses 100 to the authentication system 300, receives the iris authentication result, and transmits the iris authentication result as a response to the AR glasses 100. In addition, the authentication control unit 222 transmits the face authentication request received from the AR glasses 100 to the authentication system 300 and receives the face authentication result. The authentication control unit 222 may transmit the received face authentication result as a response to the AR glasses 100.

The speech recognition unit 223 may perform the speech recognition by distinguishing the speakers based on at least one of a volume and a speech collection direction of the conversational speech data collected by the AR glasses 100. For example, the speech recognition unit 223 may recognize a speaker of speech data having a relatively large volume as the medical worker U1 and a speaker of speech data having a relatively small volume as the patient U2 in the conversational speech data. This is because since the medical worker U1 wears the AR glasses 100, a distance to the speech collection unit 123 is shorter than that of the patient U2, and thus the speech data uttered by the medical worker U1 can be collected with a relatively large volume. In addition, the speech recognition unit 223 assumes that directivity information of the speech collection unit 123 has been set in advance. In this case, the speech recognition unit 223 may recognize a speaker of speech data whose speech collection direction is a direction directly below the speech collection unit 123 as the medical worker U1 and a speaker of speech data whose speech collection direction is a different direction as the patient U2 in the conversational speech data.

The generation unit 224 is an example of the generation unit 12 described above. The generation unit 224 generates the instruction sentence for the LLM 212 to be output in the medical record format from the text information acquired by the acquisition unit 221. Here, examples of the medical record format include, but are not limited to, a subject object assessment plan (SOAP) format. The SOAP format is a format in which medical care contents are recorded while being classified into Subject (subjective information), Object (objective information), Assessment, and Plan. Subject (subjective information) is information obtained from a content uttered by the patient U2 during the medical interview. Subject (subjective information) may include information obtained from a family member of the patient U2 or another staff of a medical institution. Object (objective information) is objective information obtained from a medical interview, an examination, an inspection, and the like. Assessment is comprehensive assessment information derived by the medical worker U1 performing analysis, interpretation, and consideration based on Subject (subjective information) and Object (objective information). Plan is information such as a future treatment policy and content for the patient U2 determined by the medical worker U1 based on Assessment. The treatment policy and content include, for example, follow-up observation, a report at the time of examination by a doctor, a medication prescription content, a prescription condition, a prescription timing, and the like.

Specifically, the generation unit 224 may generate a prompt for the LLM 212 as the instruction sentence. The prompt includes an instruction content for the LLM 212, designation of a format of an examination record as a constraint condition, a conversation content which is the text information to be processed, the utterance content of the medical worker U1 alone, and the like. The prompt may be, for example, a sentence described in a JavaScript Object Notation (JSON) format.

The display control unit 225 performs control to display, on the wearer screen 124, the past medical record information (before the current medical interview) of the patient U2 imaged by the AR glasses 100. In addition, the display control unit 225 performs control to the text information acquired by the acquisition unit 221 on the wearer screen 124. In addition, the display control unit 225 performs control to display the medical record information (output by the LLM 212 based on the instruction sentence according to the current medical interview) of the patient U2 acquired by the acquisition unit 221. Specifically, the display control unit 225 transmits a display request for each of the past medical record information, the text information, and the medical record information of the current medical interview to the AR glasses 100 together with each piece of information.

The LLM 212 does not have to be stored in the storage unit 210. In this case, the LLM 212 may be executed in an LLM system connected to the medical record support server 200 via the network N. Then, in a case where the LLM system receives the instruction sentence from the medical record support server 200 via the network N, the LLM system may generate the text information of the medical record information by inputting the instruction sentence and transmit the generated medical record information as a response to the medical record support server 200.

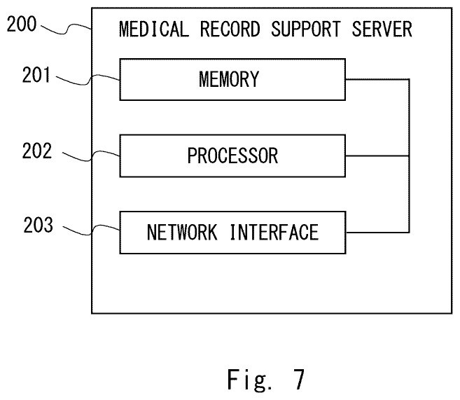

FIG. 7 is a block diagram illustrating a hardware configuration of the medical record support server 200. The medical record support server 200 includes a memory 501, a processor 202, and a network interface 203.

The memory 201 stores at least a computer program (for example, the program 211) in which at least a part of the processing of the medical record support method of the medical record support server 200 according to the present disclosure is implemented. Further, the memory 201 may store the LLM 212. Other configurations of the memory 201 are similar to those of the memory 101. The processor 202 is a control apparatus that controls each component of the medical record support server 200. The processor 202 reads and executes software (computer program) from the memory 201. As a result, the processor 202 implements the functions of the acquisition unit 221, the authentication control unit 222, the speech recognition unit 223, the generation unit 224, and the display control unit 225. That is, the processor 202 performs at least a part of the medical record support method in the medical record support server 200 according to the present disclosure. Other configurations of the processor 202 are similar to those of the processor 102. The network interface 203 has a configuration similar to that of the network interface 103.

Next, a flow in which the medical worker U1 wears the AR glasses 100 and performs the medical interview with the patient U2 to generate the medical record information will be described. As a premise, the medical worker U1 wears the AR glasses 100 before the start of the medical interview. At this time, the AR glasses 100 perform the iris authentication using the authentication system 300 based on the iris image of the medical worker U1 captured by the wearer imaging unit 121. In a case where the iris authentication has succeeded, the AR glasses 100 allow the wearer to use various functions. Hereinafter, a case where the medical worker U1 uses the function of the medical record support processing in a state of wearing the AR glasses 100 will be described.

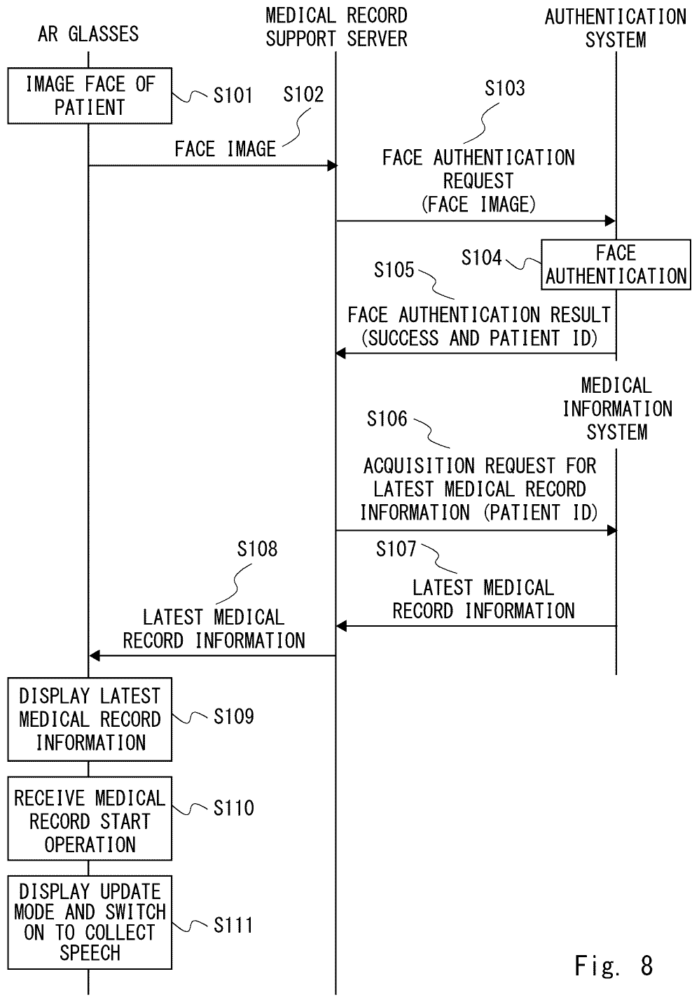

FIGS. 8, 9, and 10 are sequence charts illustrating a flow of the medical record support processing. First, the AR glasses 100 image the face of the patient U2 (S101). That is, the medical worker U1 directs the face of the medical worker U1 toward the face of the patient U2. Therefore, the AR glasses 100 image a region including the face of the patient U2 by the external imaging unit 122 and acquire the face image of the patient U2. Then, the AR glasses 100 transmit the face authentication request including the face image of the patient U2 to the medical record support server 200 via the network N (S102). Then, the medical record support server 200 transmits the received face authentication request to the authentication system 300 via the network N (S103).

Then, the authentication system 300 performs the face authentication of the patient U2 based on the received face authentication request (S104). Specifically, the authentication system 300 detects the face region from the face image included in the face authentication request, extracts a plurality of feature points indicating the face features (the eyes, the nose, the mouth, and the like) of the person from the detected face region, and calculates a distance between the respective feature points as the feature amount. Then, the authentication system 300 extracts the feature vector including a set of a plurality of calculated feature amounts as the (query) face feature information. The face authentication request may include the face feature information extracted by the AR glasses 100 or the medical record support server 200 instead of the face image.

Then, the authentication system 300 compares the query face feature information with the plurality of pieces of (master) face feature information 32 in the authentication DB 3, and calculates a matching score indicating the matching degree as the degree of coincidence. For example, the authentication system 300 may calculate the matching score to be higher as a distance between the feature vectors of the master and the query is shorter. Then, the authentication system 300 determines that the face authentication has succeeded in a case where the degree of coincidence is equal to or higher than a threshold, and determines that the face authentication has failed in a case where the degree of coincidence is lower than the threshold. The authentication system 300 includes, in the face authentication result, a determination result of the face authentication. In addition, in a case where the face authentication has succeeded, the authentication system 300 specifies the patient ID 31 associated with the face feature information 32 for which the face authentication has succeeded. Then, the authentication system 300 includes, in the face authentication result, the specified patient ID 31. Thereafter, the authentication system 300 transmits the face authentication result as a response to the medical record support server 200 via the network N (S105). Hereinafter, for convenience of explanation, it is assumed that the patient U2 has succeeded in the face authentication. Therefore, the face authentication result includes information indicating that the face authentication has succeeded and the patient ID.

Then, the medical record support server 200 receives the face authentication result from the authentication system 300 via the network N. The medical record support server 200 confirms that the patient U2 has succeeded in the face authentication from the received face authentication result, and acquires the patient ID included in the face authentication result. Then, the medical record support server 200 transmits an acquisition request for the latest medical record information including the acquired patient ID to the medical information system 400 via the network N (S106). The “latest medical record information” includes the latest medical record information among a pieces of past medical record information associated with the patient ID. Therefore, the medical record support server 200 may transmit an acquisition request for a predetermined number of pieces of medical record information from the latest medical record information among the pieces of past medical record information as the “latest medical record information”.

Then, the medical information system 400 searches the medical record DB 4 by using the patient ID included in the received acquisition request as a search key, and transmits the latest medical record information among the searched medical record information 41 and the like as a response to the medical record support server 200 (S107). Then, the medical record support server 200 transmits the received latest medical record information to the AR glasses 100 via the network N (S108). Then, the AR glasses 100 receive the latest medical record information of the patient U2 from the medical record support server 200 via the network N, and display the received latest medical record information on the wearer screen 124 (S109).

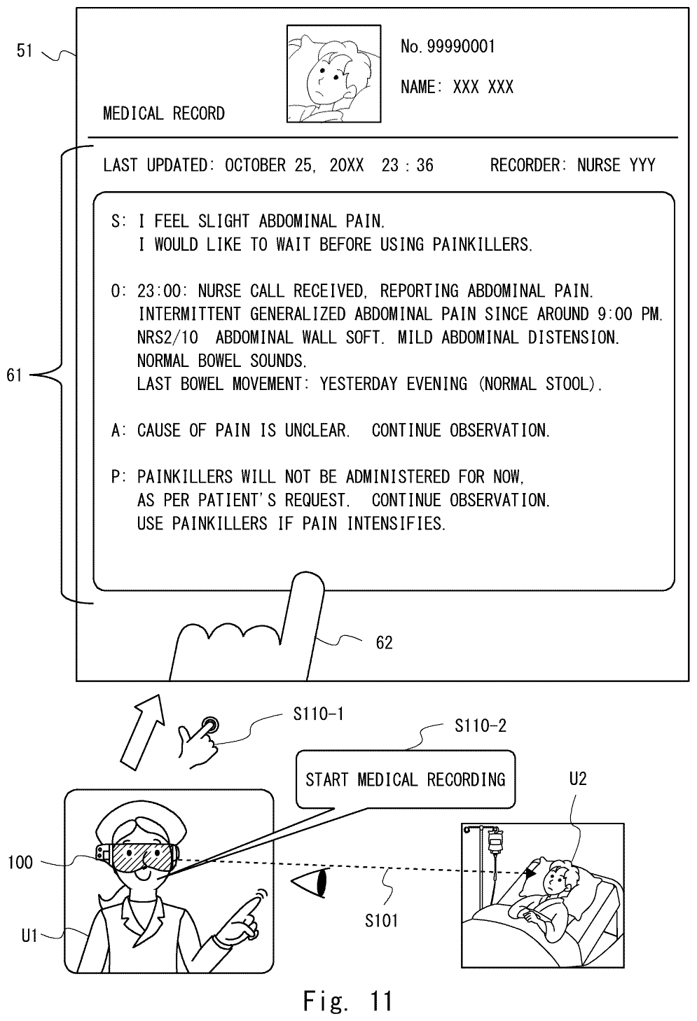

FIG. 11 is a diagram illustrating a display example of the wearer screen 124 of the AR glasses 100 at the start of the medical record support processing and a concept of a start operation. As described above, first, the medical worker U1 looks at the face of the patient U2 through the worn AR glasses 100. Therefore, the external imaging unit 122 of the AR glasses 100 images the face of the patient U2 (S101). Then, the AR glasses 100 display a medical record screen 51 on the wearer screen 124. The medical record screen 51 includes medical record information 61. The medical record information 61 is the latest information of the patient U2. In this example, the medical record information 61 is information generated based on the medical interview recently performed with the patient U2 by a nurse YYY (not illustrated) different from the medical worker U1 (nurse ZZZ). In order to generate the medical record information 61, the nurse YYY may perform the medical interview with the patient U2 in a state of wearing the AR glasses 100.

Here, before starting the medical interview with the patient U2, the medical worker U1 visually recognizes the medical record information 61 via the AR glasses 100 and then performs a medical record start operation. Then, the AR glasses 100 detect and receive the medical record start operation of the medical worker U1 (S110 in FIG. 8). Examples of the medical record start operation include, but are not limited to, the following.

(Medical record start operation)
(1: Pressing operation) First, the medical worker U1 performs an operation of pressing the medical record information 61 in the medical record screen 51 with a finger (S110-1). In this case, the AR glasses 100 detect the finger of the medical worker U1 from a captured image of the external imaging unit 122. Then, the display unit 133 of the AR glasses 100 displays a finger beam 62 at a position in the medical record screen 51, the position corresponding to a position of the detected finger. Then, in a case where the AR glasses 100 detect that the finger beam 62 is pressed at a position overlapping the medical record information 61, the AR glasses 100 detect that the medical worker U1 has been pressed the medical record information 61 with a finger.
(2: Voice command) Subsequently, the medical worker U1 utters, for example, “start medical recording” (S110-2). In this case, the AR glasses 100 detect the utterance of “start medical recording” by performing the speech recognition on the uttered speech data of the medical worker U1 collected by the speech collection unit 123.

In a case where S110-2 is detected after step S110-1, the AR glasses 100 detects that a medical record start operation A has been performed, and performs state transition to a medical record update mode.

Examples of another medical record start operation may include only the pressing operation in step S110-1 by the AR glasses 100 or only the detection of the voice command in step S110-2. Alternatively, as another medical record start operation, for example, the AR glasses 100 may image a specific information code posted near a medical treatment place of the patient U2.

After step S110 in FIG. 8, the AR glasses 100 display the update mode on the wearer screen 124, and switch the speech collection unit 123 to ON to collect the medical record (S111).

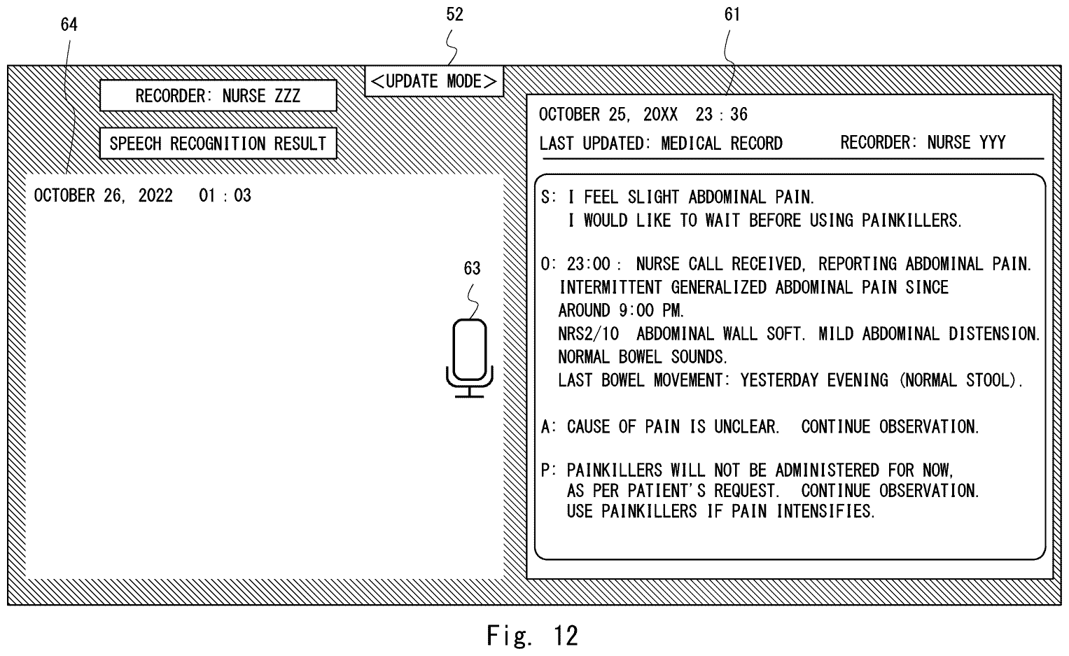

FIG. 12 is a diagram illustrating a display example of the wearer screen 124 of the AR glasses 100 immediately after the start of the medical record support processing (update mode). When the state transition to the update mode is performed, the AR glasses 100 display a medical record screen 52 on the wearer screen 124. The medical record screen 52 displays information indicating that the update mode is activated to the wearer in a visually recognizable manner. For example, “<update mode>” and “recorder: nurse ZZZ” are displayed at an upper portion of the medical record screen 52, a microphone mark 63 is displayed at the center, a speech recognition result display field 64 is displayed on a right side, the latest medical record information 61 or the like is displayed on a left side. Furthermore, a background color of the medical record screen 52 may be changed to be different from that of the medical record screen 51 in the update mode. As a result, the medical worker U1 can easily visually recognize that the current mode is the medical record update mode through the wearer screen 124 of the AR glasses 100. In addition, the medical worker U1 can visually recognize that the conversation content is added to the speech recognition result display field 64 as needed while viewing the latest medical record information 61 or the like. Therefore, the medical worker U1 can efficiently perform the medical interview. A display content indicating that the update mode is activated, a layout, and the like on the medical record screen 52 are merely examples. The medical record information 61 is the same as the content displayed on the medical record screen 51 of FIG. 11 described above. On the other hand, in the speech recognition result display field 64, only a start date of the speech recognition is displayed at this point of time, and the text information of the speech recognition result is blank.

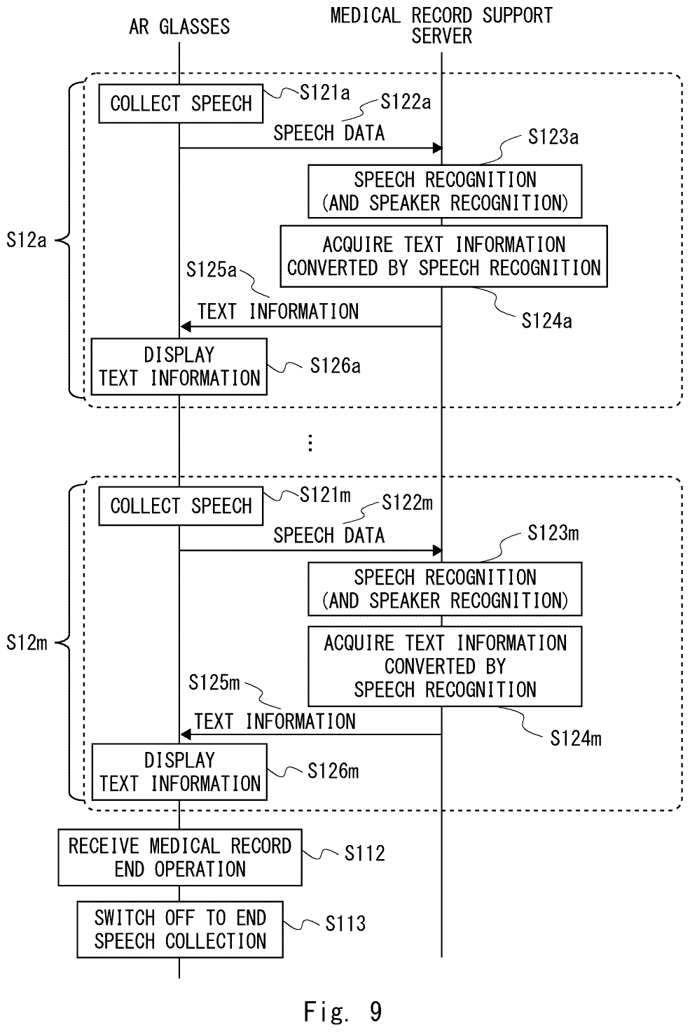

Subsequently, the medical worker U1 starts the medical interview with the patient U2. In FIG. 9, first, the AR glasses 100 cause the speech collection unit 123 to collect a speech (of the medical worker U1 or the patient U2) (S121a), and transmit the collected speech data to the medical record support server 200 (S122a). Then, the medical record support server 200 performs the speech recognition and speaker recognition on the received speech data (S123a). A known technology can be used for speech recognition processing. Furthermore, as described above, for example, the speech recognition unit 223 of the medical record support server 200 may perform the speaker recognition by recognizing a difference between the speakers based on at least one of the volume and the speech collection direction. However, in speaker recognition processing, it is not necessary to specify an individual, and it is only necessary to recognize which of a plurality of speakers the speaker is. Subsequently, the medical record support server 200 acquires the text information (and distinguishment of the recognized speaker) converted by the speech recognition (S124a). Then, the medical record support server 200 transmits the acquired text information to the AR glasses 100 (S125a). Then, the AR glasses 100 additionally display the received text information as the speech recognition result in the speech recognition result display field 64 (S126a). The flow of the processing from steps S121a to S126a is referred to as step S12a.

Thereafter, an utterance of the medical worker U1 and an utterance of the patient U2 are repeated until step S12m (m is a natural number of 2 or more) similarly to step S12a. For example, since the processing of steps S121m to S126m is equivalent to the processing of steps S121a to S126a, a detailed description thereof will be omitted.

Thereafter, the medical worker U1 performs a medical record end operation at a timing when it is determined that the medical interview has been sufficiently performed. Then, the AR glasses 100 detect and receive the medical record end operation of the medical worker U1 (S112). The medical record end operation may be an operation equivalent to the start operation described above. Then, the AR glasses 100 end displaying the update mode on the wearer screen 124 and switch the speech collection unit 123 to OFF to end the collection of the medical record (S111).

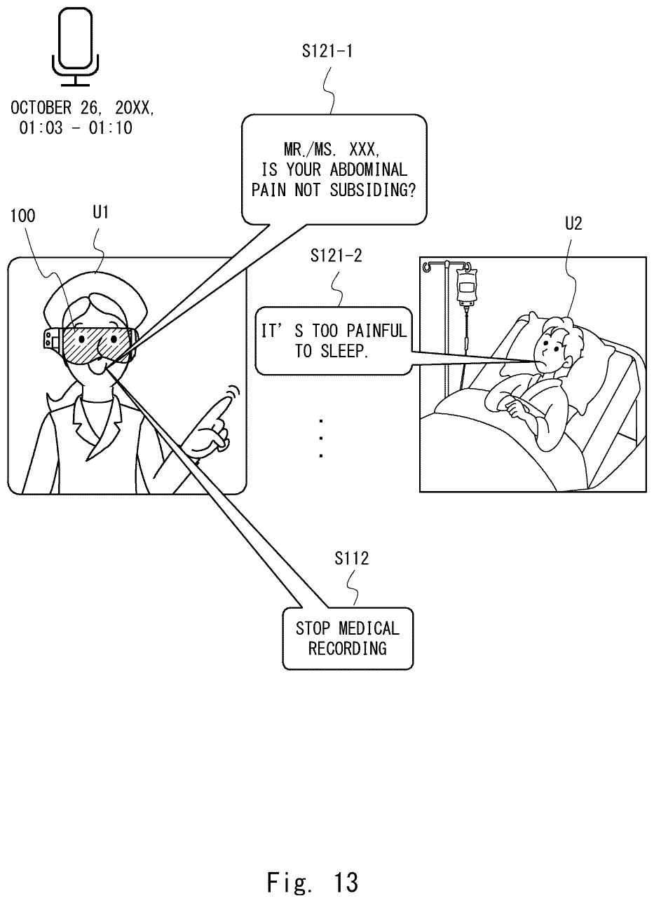

FIG. 13 is a diagram for describing the medical interview with the patient U2 by the medical worker U1 and a concept of an operation of ending the medical record support processing. FIG. 13 exemplifies the following medical interview. For example, the medical worker U1 asks the patient U2, “Mr./Ms. XXX, is your abdominal pain not subsiding?” (S121-1). Then, the patient U2 replies that “It's too painful to sleep.” (S121-2). The AR glasses 100 collect the conversation speech in the medical interview after step S121-1, and continue the processing of steps S12a to S12m in FIG. 9 described above. Thereafter, the medical worker U1 utters a voice command for the end operation “stop medical recording” (S112).

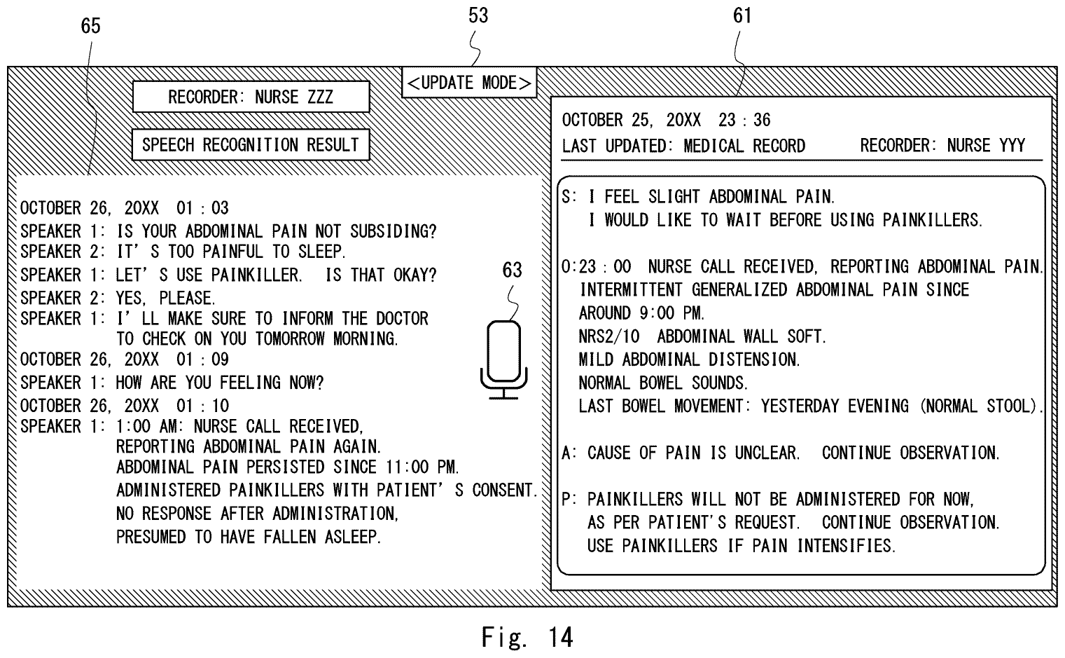

FIG. 14 is a diagram illustrating a display example of the wearer screen 124 of the AR glasses 100 during the speech recognition of the medical record support processing (update mode). A medical record screen 53 is an example in which the speech recognition result display field 64 of the medical record screen 52 is updated to a speech recognition result display field 65. That is, during the speech recognition in the update mode, the AR glasses 100 additionally display the text information obtained by the speech recognition, the identified speaker, a speech collection time, and the like in the speech recognition result display field 65 in association with each other.

The speech recognition result (text data following “October 26, 20XX 01:10 Speaker 1:”) at the end of the speech recognition result display field 65 is an example of a content obtained when the medical worker U1 confirms that there is no response from the patient U2 and then utters a summarized content based on an idea of the medical worker U1 for the medical record. That is, the AR glasses 100 perform the speech recognition not only for the conversation (exchange of utterances) between the medical worker U1 and the patient U2 in the medical interview, but also for a content uttered by the medical worker U1 alone (not an utterance toward the patient U2). In a case where the AR glasses 100 are in the update mode, the content uttered by the medical worker U1 alone may be subjected to the speech recognition and added as a target of the medical record information even before the start of the medical interview with the patient U2. As a result, a voice memo of the medical worker U1 can be added to the medical record, so that accuracy of the medical record information can be improved, and a time and effort for correction can be reduced.

After step S113 in FIG. 9, the AR glasses 100 transmit a generation request for the medical record information to the medical record support server 200 via the network N (S201). The AR glasses 100 may transmit an update mode end notification or the like to the medical record support server 200 instead of the generation request.

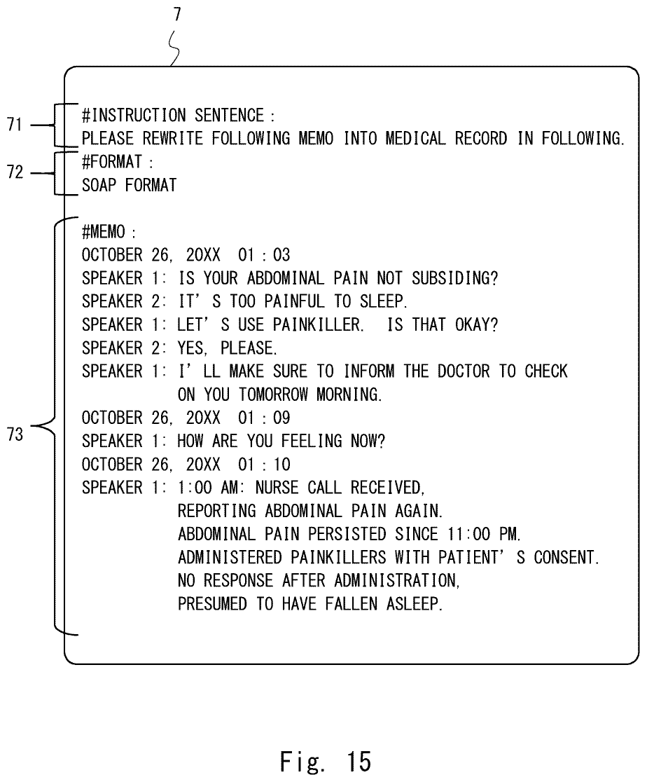

In response to the reception of the generation request or the like, the medical record support server 200 generates the prompt for the LLM 212 by using a text information group converted in the update mode (S202). FIG. 15 is a diagram illustrating an example of text conversion and a prompt 7 generated based on the speech recognition result. The prompt 7 is an example of the instruction sentence generated according to an input format of the LLM 212. The prompt 7 includes an instruction sentence 71, a constraint condition 72, and speech recognition text information 73. The instruction sentence 71 is text data expressed in a sentence so as to cause the LLM 212 to convert the speech recognition text information 73 into the medical record information and summarize the speech recognition text information 73 based on the constraint condition 72. The constraint condition 72 is an example in which the SOAP format is designated as the format of the medical record. The speech recognition text information 73 is text information displayed in the speech recognition result display field 64 in FIG. 14.

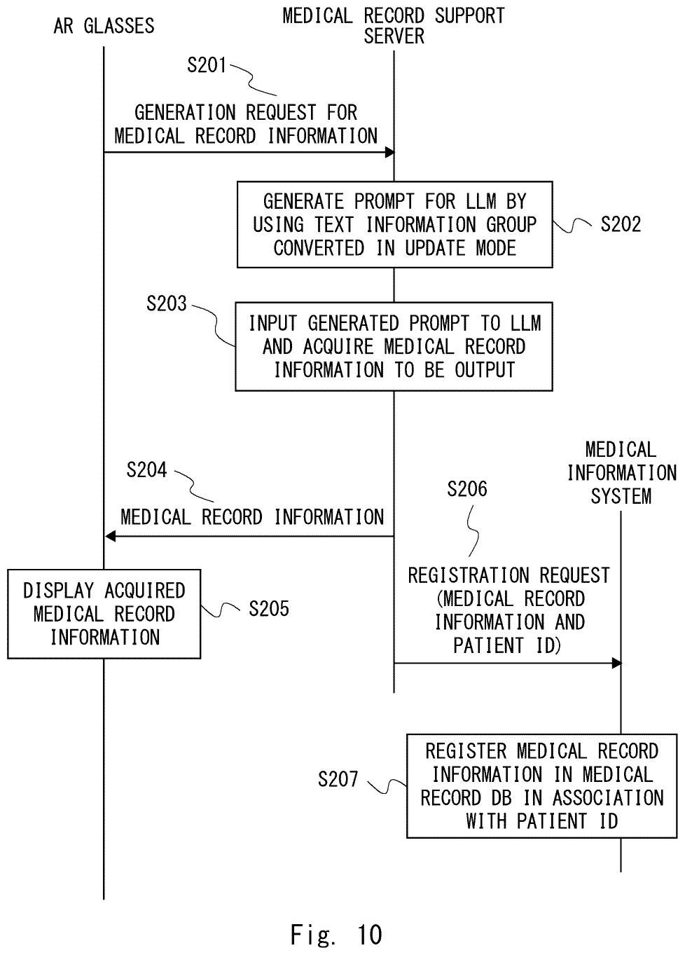

After step S202 in FIG. 10, the medical record support server 200 inputs the generated prompt to the LLM 212 and acquires the medical record information output from the LLM 212 (S203). That is, the LLM 212 converts and summarizes the speech recognition text information 73 into the SOAP format based on the instruction sentence 71 and the constraint condition 72 of the input prompt 7 by using a deep-learned parameter group, and generates and outputs the text information.

Then, the medical record support server 200 transmits the medical record information acquired from the LLM 212 to the AR glasses 100 via the network N (S204). Then, the AR glasses 100 display the medical record information acquired from the medical record support server 200 via the network N on the wearer screen 124 (S205).

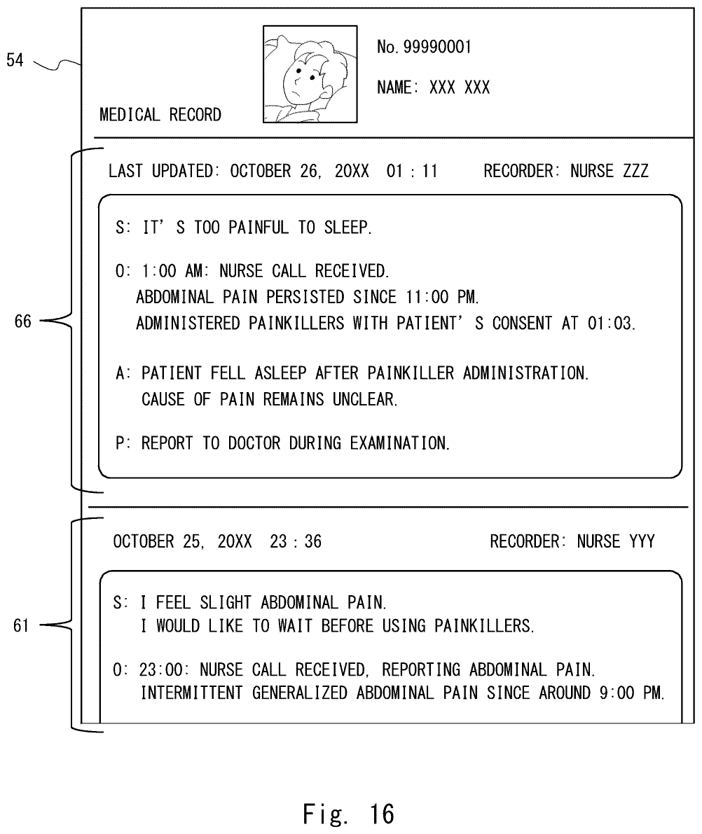

FIG. 16 is a diagram illustrating a display example of the latest medical record output by the LLM 212 from the generated prompt 7. A medical record screen 54 includes medical record information 66 and a part of the medical record information 61. The medical record information 66 is the latest medical record information in the current medical interview with the patient U2 by the medical worker U1 (nurse ZZZ). The medical record information 61 is the latest medical record information created by another nurse YYY before the current medical interview.

Furthermore, after step S203 in FIG. 10, together with step S204, the medical record support server 200 transmits a registration request for the medical record information to the medical information system 400 via the network N (S206). At this time, the registration request includes the patient ID of the patient U2 received in step S105 and the medical record information acquired in step S203. Then, the medical information system 400 registers the received medical record information in the medical record DB 4 in association with the patient ID (S207).

In the above description, the face authentication of the patient U2 is performed in order to display the latest medical record information on the AR glasses 100, but a method of displaying the latest medical record information is not limited thereto. As a method of displaying the past medical record information of a specific patient, a function of existing medical AR glasses or a function of a medical institution system may be used.

Furthermore, the medical record support server 200 may have the function of the medical information system 400. In this case, the storage unit 210 of the medical record support server 200 further stores the medical record DB 4. Furthermore, the medical record support server 200 may cause an external system to perform the speech recognition processing by the speech recognition unit 223 to acquire the speech recognition result. Alternatively, the AR glasses 100 may perform the speech recognition processing. For example, the AR glasses 100 may have the function of the speech recognition unit 223, and perform the speech recognition by distinguishing the speakers based on at least one of the volume and the speech collection direction in the conversational speech data collected by the speech collection unit 123. In this case, communication between the AR glasses 100 and the medical record support server 200 in steps S12a to S12m in FIG. 9 becomes unnecessary, and thus, a communication load can be reduced.

Here, problems to be solved by the medical record support system and the like according to the present disclosure will be described again. First, in a medical site, it is common that one medical worker, particularly a nurse, serves a large number of patients and serves a common patient with other medical workers. Therefore, the medical worker is busy, as a result of which creation of the medical record such as the nursing record is delayed, and the medical records are collectively created at the end of working hours in many cases. Therefore, a burden on the body and mind of the medical worker is large, and a lot of time is required for work of creating the medical records. Therefore, there is a problem that it is difficult for the medical worker, particularly a nurse, to concentrate on the treatment, medical interview, and the like for the patient, which are the original work. In addition, in a case where a nurse or the like creates a nursing record or the like while relying on memory after all the medical interview or the like is completed, there is also a problem that a significant burden is placed on ensuring accuracy of the medical record. That is, since the medical worker needs to accurately and quickly create the medical record, there is a problem that a workload tends to increase.

Therefore, by using the medical record support system or the like according to the present disclosure, the medical worker such as a nurse can quickly record the medical record information by utilizing a speech input to the worn eyewear-type wearable terminal. In particular, since the medical worker U1 wears the AR glasses 100 and performs the medical interview with the patient U2, the medical record is not handwritten and is not created by performing an input operation to an information terminal at the time of the medical interview. That is, the medical worker U1 can perform the medical interview with the patient U2 in a state where both hands are not occupied. Therefore, the medical worker U1 can continue the medical interview while performing some work on the patient U2, and can efficiently create the medical record.

Therefore, it is possible to reduce the workload of the medical worker such as a nurse, and it is easy to concentrate on the care of the patient. Therefore, it is possible to implement a workplace where the medical worker can work actively and improve the quality of medical care and patient satisfaction. Furthermore, it is also possible to support a manager of a medical institution who wants to care for the medical worker such as a nurse.

In addition, since the medical record information is generated and displayed immediately after the medical interview, the medical worker can easily confirm and finely correct the medical record information. Therefore, the accuracy and authenticity of the medical record information can be ensured. Then, a time for creating the medical record can be significantly shortened.

In addition, the AR glasses 100 transitions to the update mode by the start operation of the medical worker U1 after the medical worker U1 wears the AR glasses 100. Therefore, the AR glasses 100 can prevent erroneous speech collection, speech recognition, text conversion, and the like for the utterances of the medical worker U1 and other persons before and after the update mode. Therefore, it is possible to prevent an unintended cancellation operation or the like for the medical record support processing by the medical worker U1. Therefore, the burden of the work of creating the medical record by the medical worker U1 is further reduced.

### Third Embodiment

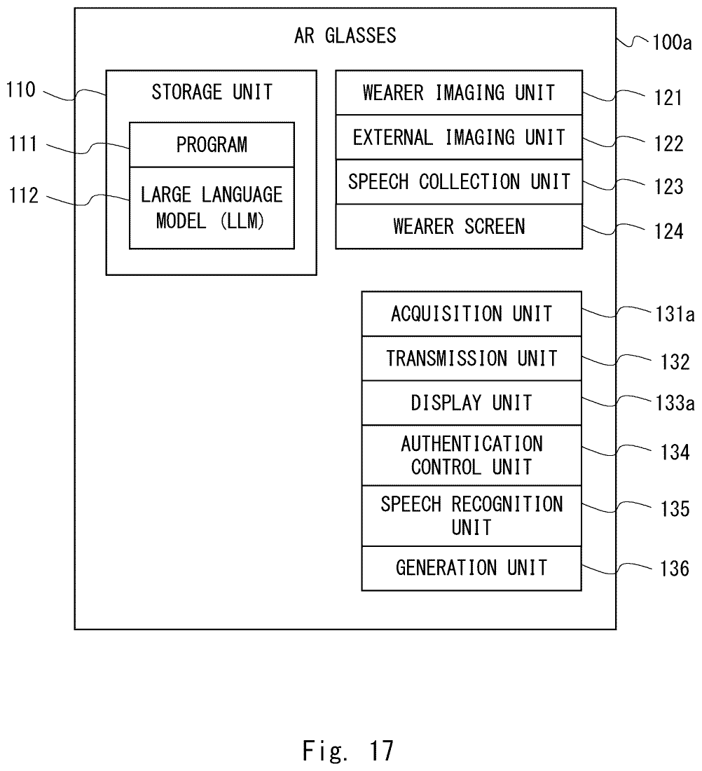

Hereinafter, a case where the eyewear-type wearable terminal alone performs the processing of the medical record support method according to the present disclosure without using the medical record support server will be described. FIG. 17 is a block diagram illustrating a configuration of AR glasses 100a. The AR glasses 100a are an eyewear-type wearable terminal worn by a medical worker during a medical interview. The AR glasses 100a are different from the AR glasses 100 of FIG. 4 described above in that a program 111, an LLM 112, an authentication control unit 134, a speech recognition unit 135, and a generation unit 136 are added, and an acquisition unit 131a and a display unit 133a are changed. Therefore, a description of the configuration common to the AR glasses 100 is omitted below. Furthermore, the hardware configuration of the AR glasses 100a is similar to that of FIG. 5 described above.

The storage unit 110 corresponds to the memory 101 of FIG. 5 described above. A program 111 is obtained by changing the computer program stored in the memory 101 described above. The program 111 is a computer program in which the processing of the medical record support method according to the present disclosure is implemented. The LLM 112 is a large language model equivalent to the LLM 212 of FIG. 6 described above. The LLM 112 does not have to be stored in the storage unit 110. In this case, the LLM 112 may be executed in an LLM system connected to the AR glasses 100a via a network N. Then, in a case where the LLM system receives an instruction sentence from the AR glasses 100a via the network N, the LLM system may generate text information of medical record information by inputting the instruction sentence and transmit the generated medical record information as a response to the AR glasses 100a.

The acquisition unit 131a is an example of the first acquisition unit 11 and the second acquisition unit 13 described above. The acquisition unit 131a acquires text information converted based on conversational speech data collected by a speech collection unit 123. In addition, the acquisition unit 131a has the function of the acquisition unit 131 described above, and at the same time, acquires an authentication result and the medical record information by communication with an authentication system 300 and a medical information system 400 like the acquisition unit 221.

The display unit 133a displays the medical record information acquired by the acquisition unit 131a on a wearer screen 124. In addition, the display unit 133a may further display the text information acquired by the acquisition unit 131a on the wearer screen 124. The display unit 133a may further display the past medical record information of a patient imaged by an external imaging unit 122 on the wearer screen 124.

The authentication control unit 134 is similar to the authentication control unit 222 described above. The speech recognition unit 135 may perform speech recognition by distinguishing speakers based on at least one of a volume and a speech collection direction of the conversational speech data collected by the speech collection unit 123. Therefore, the acquisition unit 131a acquires the text information converted from a recognition result of the speech recognition in which the speakers are distinguished by the speech recognition unit 135. The generation unit 136 is similar to the generation unit 224 described above.

Furthermore, a flow of medical record support processing using the AR glasses 100a can be implemented by appropriately changing the above-described second embodiment. Specifically, the communication between the medical record support server 200, the authentication system 300, and the medical information system 400 in FIG. 6 is replaced with communication between the AR glasses 100a, the authentication system 300, and the medical information system 400. Furthermore, the communication between the AR glasses 100 and the medical record support server 200 in FIG. 9 is omitted, and the AR glasses 100a perform the processing of steps S123a and S124a. Furthermore, the communication between the AR glasses 100 and the medical record support server 200 in FIG. 10 is omitted, and the AR glasses 100a perform the processing of steps S202, S203, and S206.

As described above, the third embodiment can also achieve the same effects as those of the second embodiment described above.

### Other Embodiments

The medical record support server 200 described above may incorporate some or all of the configurations and functions of the authentication system 300 and the medical information system 400.

While the present disclosure has been particularly shown and described with reference to embodiments thereof, the present disclosure is not limited to these embodiments. It will be understood by those of ordinary skill in the art that various changes in form and details may be made therein without departing from the sprit and scope of the present disclosure as defined by the claims. And each embodiment can be appropriately combined with at least one of embodiments.

Each of the drawings or figures is merely an example to illustrate one or more embodiments. Each figure may not be associated with only one particular embodiment, but may be associated with one or more other embodiments. As those of ordinary skill in the art will understand, various features or steps described with reference to any one of the figures can be combined with features or steps illustrated in one or more other figures, for example to produce embodiments that are not explicitly illustrated or described. Not all of the features or steps illustrated in any one of the figures to describe an example embodiment are necessarily essential, and some features or steps may be omitted. The order of the steps described in any of the figures may be changed as appropriate.

A (The) program can be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g. magneto-optical disks), CD-ROM (compact disc read only memory), CD-R (compact disc recordable), CD-R/W (compact disc rewritable), and semiconductor memories (such as mask ROM, PROM (programmable ROM), EPROM (erasable PROM), flash ROM, RAM (random access memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g. electric wires, and optical fibers) or a wireless communication line.

Some or all of the above-described embodiments may be described as in the following Supplementary Notes, but are not limited to the following Supplementary Notes.

### (Supplementary Note A1)

A medical record support apparatus including:
a first acquisition unit configured to acquire text information of an utterance content converted by speech recognition in which speakers are distinguished for conversational speech data between a medical worker and a patient during a medical interview;
a generation unit configured to generate an instruction sentence for a language model to be output in a medical record format from the text information; and
a second acquisition unit configured to acquire medical record information of the patient by inputting the instruction sentence to the language model.

### (Supplementary Note A2)

The medical record support apparatus according to Supplementary Note A1, in which
the medical record support apparatus is an eyeglass-type wearable terminal worn by the medical worker during the medical interview,
the first acquisition unit is configured to acquire the converted text information based on the conversational speech data collected by the eyewear-type wearable terminal, and
the medical record support apparatus further includes a display unit configured to display the medical record information acquired by the second acquisition unit on a wearer screen of the eyewear-type wearable terminal.

### (Supplementary Note A3)

The medical record support apparatus according to Supplementary Note A2, in which the display unit is configured to further display the text information acquired by the first acquisition unit on the wearer screen.

### (Supplementary Note A4)

The medical record support apparatus according to Supplementary Note A2 or A3, in which the display unit is configured to further display past medical record information of the patient imaged by the eyewear-type wearable terminal on the wearer screen.

### (Supplementary Note A5)

The medical record support apparatus according to any one of Supplementary Notes A2 to A4, further including a speech recognition unit configured to perform the speech recognition in which the speakers are distinguished based on at least one of a volume and a speech collection direction of the conversational speech data collected by the eyewear-type wearable terminal,
wherein the first acquisition unit acquires the text information converted from a recognition result of the speech recognition in which the speakers are distinguished by the speech recognition unit.

### (Supplementary Note A6)

The medical record support apparatus according to Supplementary Note A1, in which
the first acquisition unit is configured to acquire the converted text information based on the conversational speech data collected by the eyewear-type wearable terminal worn by the medical worker during the medical interview, and
the medical record support apparatus further includes a display control unit configured to perform control to display the medical record information on a wearer screen of the eyewear-type wearable terminal.

### (Supplementary Note A7)

The medical record support apparatus according to Supplementary Note A6, in which the display control unit is configured to perform control to further display the text information acquired by the first acquisition unit on the wearer screen.

### (Supplementary Note A8)

The medical record support apparatus according to Supplementary Note A6 or A7, in which the display control unit is configured to perform control to further display past medical record information of the patient imaged by the eyewear-type wearable terminal on the wearer screen.

### (Supplementary Note A9)

The medical record support apparatus according to any one of Supplementary Notes A6 to A8, further including a speech recognition unit configured to perform the speech recognition in which the speakers are distinguished based on at least one of a volume and a speech collection direction of the conversational speech data collected by the eyewear-type wearable terminal,
wherein the first acquisition unit acquires the text information converted from a recognition result of the speech recognition in which the speakers are distinguished by the speech recognition unit.

### (Supplementary Note A10)

The medical record support apparatus according to any one of Supplementary Notes A1 to A9, in which the text information includes text information converted as an utterance content of the medical worker by speech recognition for uttered speech data of the medical worker before and after the medical interview.

### (Supplementary Note A11)

The medical record support apparatus according to any one of Supplementary Notes A1 to A10, in which
the medical worker is a nurse, and
the medical record information includes information of a nursing record. (Supplementary Note A12)

The medical record support apparatus according to any one of Supplementary Notes A1 to A11, in which the medical record format is a subject object assessment plan (SOAP) format.

### (Supplementary Note B1)

A medical record support system including:
an eyewear-type wearable terminal worn by a medical worker during a medical interview; and
an information processing apparatus communicably connected to the eyewear-type wearable terminal, in which
the information processing apparatus includes:
a first acquisition unit configured to acquire text information of an utterance content converted by speech recognition in which speakers are distinguished for conversational speech data between the medical worker and a patient during the medical interview, the conversational speech data being collected by the eyewear-type wearable terminal;
a generation unit configured to generate an instruction sentence for a language model to be output in a medical record format from the text information; and
a second acquisition unit configured to acquire medical record information of the patient by inputting the instruction sentence to the language model, and
the eyewear-type wearable terminal is configured to display the medical record information acquired by the second acquisition unit on a wearer screen.

### (Supplementary Note C1)

A medical record support method executed by a computer, the medical record support method including:
acquiring text information of an utterance content converted by speech recognition in which speakers are distinguished for conversational speech data between a medical worker and a patient during a medical interview;
generating an instruction sentence for a language model to be output in a medical record format from the text information; and
acquiring medical record information of the patient by inputting the instruction sentence to the language model.

### (Supplementary Note D1)

A medical record support program causing a computer to perform:
first acquisition processing of acquiring text information of an utterance content converted by speech recognition in which speakers are distinguished for conversational speech data between a medical worker and a patient during a medical interview;
generation processing of generating an instruction sentence for a language model to be output in a medical record format from the text information; and
second acquisition processing of acquiring medical record information of the patient by inputting the instruction sentence to the language model.

Some or all of the elements (for example, the configurations and functions) described in Supplementary Notes A2 to A12 dependent on Supplementary Note A1 {e.g. apparatus} can also be dependent on Supplementary Note B1 {e.g. system}, Supplementary Note C1 {e.g. method}, and Supplementary Note D1 {e.g. program} by the same dependency relationship as Supplementary Notes A2 to A12. Some or all of the elements described in any supplementary notes may be applied to various hardware, software, recording means for recording software, systems, and methods.

What is claimed is:

1. A medical record support apparatus comprising:
at least one memory; and
at least one processor coupled to the at least one memory,
wherein the at least one processor is configured to:
acquire text information of an utterance content converted by speech recognition in which speakers are distinguished for conversational speech data between a medical worker and a patient during a medical interview;
generate an instruction sentence for a language model to be output in a medical record format from the text information; and
acquire medical record information of the patient by inputting the instruction sentence to the language model.

2. The medical record support apparatus according to claim 1, wherein
the medical record support apparatus is an eyewear-type wearable terminal worn by the medical worker during the medical interview, and
the at least one processor is configured to:
acquire the converted text information based on the conversational speech data collected by the eyewear-type wearable terminal; and
display the acquired medical record information on a wearer screen of the eyewear-type wearable terminal.

3. The medical record support apparatus according to claim 2, wherein
the at least one processor is configured to perform the speech recognition in which the speakers are distinguished based on at least one of a volume and a speech collection direction of the conversational speech data collected by the eyewear-type wearable terminal, and
the at least one processor is configured to distinguish the speakers and acquire the text information converted from a recognition result of the speech recognition.

4. The medical record support apparatus according to claim 1, wherein the at least one processor is configured to:
acquire the converted text information based on the conversational speech data collected by the eyewear-type wearable terminal worn by the medical worker during the medical interview; and
perform control to display the medical record information on a wearer screen of the eyewear-type wearable terminal.

5. The medical record support apparatus according to claim 4, wherein
the at least one processor is configured to perform the speech recognition in which the speakers are distinguished based on at least one of a volume and a speech collection direction of the conversational speech data collected by the eyewear-type wearable terminal, and
the at least one processor is configured to distinguish the speakers and acquire the text information converted from a recognition result of the speech recognition.

6. The medical record support apparatus according to claim 1, wherein the text information includes text information converted as an utterance content of the medical worker by speech recognition for uttered speech data of the medical worker before and after the medical interview.

7. The medical record support apparatus according to claim 1, wherein
the medical worker is a nurse, and
the medical record information includes information of a nursing record.

8. A medical record support system comprising:
an eyewear-type wearable terminal worn by a medical worker during a medical interview; and
an information processing apparatus communicably connected to the eyewear-type wearable terminal, wherein
the information processing apparatus includes:
at least one apparatus memory; and
at least one apparatus processor coupled to the at least one apparatus memory,
the at least one apparatus processor is configured to:
acquire text information of an utterance content converted by speech recognition in which speakers are distinguished for conversational speech data between the medical worker and a patient during the medical interview, the conversational speech data being collected by the eyewear-type wearable terminal;
generate an instruction sentence for a language model to be output in a medical record format from the text information; and
acquire medical record information of the patient by inputting the instruction sentence to the language model,
the eyewear-type wearable terminal includes:
at least one terminal memory; and
at least one terminal processor coupled to the at least one terminal memory, and
the at least one terminal processor is configured to display the medical record information on a wearer screen.

9. A medical record support method executed by a computer, the medical record support method comprising:
acquiring text information of an utterance content converted by speech recognition in which speakers are distinguished for conversational speech data between a medical worker and a patient during a medical interview;
generating an instruction sentence for a language model to be output in a medical record format from the text information; and
acquiring medical record information of the patient by inputting the instruction sentence to the language model.
